(19) United States
(12) Patent Application Publication
(10) Pub. No.: US 2015/0338380 A1
(43) Pub. Date: Nov. 26, 2015

(54) Assessing Corrosion Damage in Post-Tensioned Concrete Structures Using Acoustic Emission

(71) Applicant: University of South Carolina, Columbia, SC (US)

(72) Inventors: Paul Henry Ziehl, Irmo, SC (US); Mohamed ElBatanouny, Columbia, SC (US); Aditya Appalla, Columbia, SC (US); Jese Mangual, Columbia, SC (US)

(21) Appl. No.: 14/718,572

(22) Filed: May 21, 2015

Related U.S. Application Data
(60) Provisional application No. 62/001,291, filed on May 21, 2014.

(51) Int. Cl. G01N 29/14; G01N 33/38

(52) U.S. Cl. CPC G01N 29/14; G01N 33/383; G01N 2291/0232; G01N 2291/044

(57) ABSTRACT

Methods are provided for detecting damage in a post-tensioned concrete specimen due to corrosion. In one embodiment, the method comprises mounting at least two piezoelectric sensors onto the surface of the post-tensioned concrete specimen; receiving acoustic emission signals at the piezoelectric sensors; recording the acoustic emission signals with associated parameters and waveforms; and filtering the data for example using Root Mean Square data and the average frequency data.

### PRIORITY INFORMATION

The present application claims priority to U.S. Provisional Patent Application Ser. No. 62/001,291 titled “Assessing Corrosion Damage in Post-Tensioned Concrete Structures Using Acoustic Emission” of Ziehl, et al. filed on May 21, 2014; the disclosure of which is incorporated by reference herein.

### BACKGROUND: INTRODUCTION TO PRESTRESSING

Concrete is a brittle material with compressive strength values far exceeding those for tensile strength. Prestressing is a method of construction of concrete structures where internal tensile stress is applied so that the concrete remains in compression when external loads are applied. One of the earliest examples of prestressing is the construction of wooden barrels by force fitting of metal bands. In 1904 Eugene Freyssinet pioneered the method of prestressing which has now become a major construction practice for concrete structures.

Prestressed concrete offers many advantages in comparison to passively reinforced concrete structures. Some advantages of prestressed structural members are a) the concrete is either free from tensile stress or very low tensile stress is allowed; b) the uncracked moment of inertia can be utilized, thus saving on material costs; c) improved resistance to shear due to the imposed compressive stress; and d) reduced on-site construction time. The use of high strength concrete and steel results in lighter member cross-sections and therefore reduced dead loads. These features also contribute to improved durability of the structure under aggressive environmental conditions. Standardized prestressed bridge girders between 9.5 m (30 ft) and 27.5 m (90 ft) are significantly more economical than steel or reinforced concrete girders, and the trend is to utilize prestressed for girders of spans up to 46 m (150 ft).

Prestressing is subdivided into two categories; Pretensioning and Post-Tensioning [PT]. In pretensioning, the tendons are laid first and stressed after the concrete is placed. In post-tensioning [PT], tendons are laid in ducts and are stressed after concrete has been placed and hardened. Post-tensioning [PT] can be further divided into internal and external post-tensioning. In external post-tensioning [PT], the strands are laid outside the structure, whereas for internal post-tensioning the ducts pass within the structure and are covered by the concrete.

For internal post-tensioning [PT], the ducts can be grouted which are known as bonded post-tensioning [PT] or in some cases not grouted, also known as unbonded post-tensioning. Bonded post-tensioned ducts are generally 2.5 times the diameter of the strand and generally carry multiple strands. On the other hand unbonded post-tensioned ducts are the size of the strand and have the provision to carry only a single stand. In unbonded post-tensioning, the strands are greased before passing them into ducts and tensioning. For an example, unbonded post-tensioning has its applications in slabs where the thickness of the slab is not sufficient to accommodate a bigger duct, while bonded post-tensioning is use in case of large scale structures like bridge girders or decks. Since the 1960's, post-tensioning [PT](internal and external PT) has become a popular and accepted method for construction for bridges across the United States and throughout out the world. Ease of construction, higher strengths, and low costs have contributed to this popularity.

Each of the methods of post-tensioning has advantages and disadvantages, as listed in Table 1.1.

TABLE 1.1
Advantages & Disadvantages of Internal and External PT

| | |
|---|---|
| Advantages | Disadvantages |

Internal Post—Tensioning

| | |
|---|---|
| 1. Less prone to damage. | 1. Rigorous maintenance |
| 2. Strands are laid as per the | and repair procedures. |
| bending moment diagram; | Some locations are |
| hence the loads are properly | completely inaccessible. |
| balanced. | |

External Post—Tensioning

| | |
|---|---|
| 1. Smaller sections required so | 1. More prone to damage |
| reduced dead weight. | because the strands are |
| 2. Reduced material, therefore | located outside the |
| lower costs. | structure. |
| 3. Reduced maintenance and | |
| repair. | |

Both of these methods of PT were developed at the same time, and are extensively used for post-tensioning. In some cases like bridges, mixed post-tensioning is also preferred wherein both internal and external post-tensioning is adopted. In both external and internal PT, the tendons pass through either metal, High Density Poly Ethylene [HDPE], or poly propylene ducts to transfer stresses from the strands to the concrete.

Initially, it was believed that post-tensioning was a low maintenance method of construction. However, the sudden failure of the Bickton Meadows foot bridge in 1967 and the Ynys-y-Gwas bridge in 1985 raised questions about the durability of this construction method.

Of 600,000 bridges in the U.S., roughly 18% are prestressed. Some of these are exhibiting corrosion in the prestressing strands. Major factors for corrosion include carbonation, high chlorides in the grout mix, ingress of chlorides due to the environment (deicing salts or location of the bridge in coastal regions), air pockets in ducts, bleed water, and faulty construction practices. High corrosion levels have been found in Florida due to the coastal climate. Some of the major bridges in that area include the Sunshine Skyway Bride, the Niles Channel Bridge, and the Mid Bay Bridge. The Mid Bay Bridge was opened in 1993, and by 2000 the corrosion levels in some tendons were so high that 11 tendons in the bridge had to be replaced. The same was observed in the Skyway Bridge and the Niles channel bridge.

It was also observed that cracking of the ducts due to faulty construction practices or slipping of the ducts at deviator blocks or anchorages in the Mid Bay Bridge left the strand exposed to the environment, thereby accelerating the corrosion process. Further boroscopic inspections revealed corrosion in many other tendons.

In May 2000, a 24.5 m (80-ft) section of the Lowes Motor Speedway Bridge in North Carolina collapsed killing and injuring several people. Investigations have revealed that the cause of the collapse was corrosion of the strands. This was in turn due to the use of a grout with excess calcium chloride during the general repair and maintenance procedures of the bridge. In 2011, the FHWA released a list of 35 post-tensioned bridges across the U.S. that are experiencing corrosion in their strands due to excess chlorides in the grout mix, and the list is expected to increase.

Some of the basic terminology related to prestressing is provided below for convenience:

Strand—A high tensile cable made of steel, typically comprised of seven individual wires, with six wires helically wound around a single central wire. Strands for post-tensioning purpose are typically Grade 1,860 MPa (270 ksi) confirming to ASTM A416 [1]. It is commonly available in two sizes, 12.7 mm (0.5 in) and 15.24 mm (0.6 in) diameter. These strands are manufactured by a cold drawing process which makes them resistant to corrosion.

Tendons—A group of strands that are connected to the same anchor and located in the same duct is known as a tendon. A typical post-tensioned bridge segment has many groups of tendons laid in various positions.

Ducts—Hollow structures made either from High Density Poly Ethylene [HDPE] or metal, through which tendons pass. Ducts can be laid within or outside the structure. The external surface can be corrugated or smooth depending on where the ducts are being laid. For internal post-tensioning, corrugated ducts are used as they provide improved bond strength. The diameter of the ducts is generally 2.5 times the diameter of the tendons.

Grout—A mix of cement, fine aggregates, and admixtures in a certain proportion which meets the requirements of ASTM C953, ASTM C942, ASTM C1202, ASTM C1090, ASTM C939, and/or ASTM C940. Water cement ratio is generally limited to 0.45 to minimize bleeding. These grouts can be mixed in the field per an approved design or can be pre-bagged by a manufacturer.

Void—A void is an internal air pocket in the grouted ducts. Voids occur due to improper grouting practices or bleeding. They trap moisture and can lead to corrosion of the tendon or strand.

Anchorage—A device used to enable the tendon to impart and maintain the desired range of stress in the concrete.

Pretensioning—A method of prestressing in which the tendons are tensioned before the concrete is placed.

Post-tensioning—A method of prestressing wherein the strands are stressed after hardening of the concrete.

Full Prestressing—Prestressed concrete in which tensile stresses in the concrete are entirely negated at working loads by having sufficiently high prestress in the member.

Partial Prestressing—The degree of prestress applied to concrete in which tensile stresses to a limited magnitude are permitted in concrete under working loads.

In summary, the method of construction of concrete structures where highly stressed strands passing through high density polyethylene [HDPE] or poly propylene [PP] ducts in a concrete structure to transfer the stresses from the strands to concrete is known as Post-Tensioning [PT]. Some of the advantages of post-tensioned structures are higher strength, smaller cross-sections, low or no cracking, ease of construction, and related economic viability. Post-Tensioned structures are either bonded or unbonded. In bonded post-tensioning, the strands are passed through ducts that are later filled with a cement based grout whereas the ducts of unbonded post-tensioned structures are left ungrouted. Bonded post-tensioning ducts are generally 2.5 times the diameter of the tendon and are used in structures with bigger cross-sections, while unbonded post-tensioned ducts have the provision to pass only a single strand, and are used in structures with smaller cross-section where there is a limitation for using bigger size ducts. Further in this paper only bonded post-tensioning is discussed.

Post-tensioned structures are categorized into two types; external and internal. In external post-tensioning, the strands are covered in grouted ducts that are laid outside the concrete structure, whereas internally post-tensioned structures have strands in grouted ducts placed within the concrete itself.

Several bridges built using post-tensioning method of construction are experiencing damage in their strands due to corrosion. Corrosion of the strands is an irreversible process that compromises the strength, safety and serviceability of the bridge. Further the corrosion by-products are expansive in nature and cause cracking of grout or concrete around the strand. The high alkalinity of the cement grout, with pH values generally ranging between 12 and 13, serves to protect the strands from corrosion by forming a microscopic layer of oxide over the surface of the reinforcement which is known as passivation. This passivating layer of oxide may be broken down by the ingress of chlorides into the ducts due to deicing salts, faulty construction practice or location of bridges near coastal areas.

In some cases, unusually high levels of chlorides have been observed in the grout itself that is used for post-tensioning purposes due to improper manufacturing processes. The chloride levels detected in some post-tensioned structures have exceeded the maximum permissible chloride limit of 0.08% by weight of cement material as prescribed by FHWA to a very high percentage. A very recent example is the Sika 300 PT grout which was used by many state Departments of Transportation across the U.S. High chloride levels were observed in this grout, which were up to 400% (four times) the maximum permissible limit causing deterioration of the prestressing strands in the bridges for which it was used. The existence of air voids in the ducts due to bleed water and carbonation are other major causes for corrosion of the prestressing strands.

External PT structures may be likely to corrode faster due to exposure to the environment, and the corrosion process is mostly due to deicing salts or harsh environment. Internal PT structures are less susceptible to corrosion due to atmospheric conditions as the strands are encased in the duct which is covered with a thick layer of concrete. However, the presence of chlorides in the grout mix initiates corrosion of the strands in internal PT structures and its detection and repair is difficult due to the concrete cover. On the other hand detecting corrosion in external PT systems is more readily accomplished.

### BACKGROUND: INTRODUCTION TO ACOUSTIC EMISSION MONITORING

As per ASTM E1316, Acoustic Emission [AE] is defined as ‘the class of phenomenon whereby transient elastic waves are generated by rapid release of energy from localized sources within a material, or the transient waves so generated.”

When a material is stressed, cracked or deformed, the internal particles realign themselves and release energy in the form of stress waves. These stress waves can be detected by piezoelectric sensors which convert this energy into electrical signals. Each signal has properties that can be analyzed.

Some of detectable sources are crack growth, dislocations, corrosion, friction, leaks, and cavitations. Some advantages of AE include:
a. The energy detected comes from within the material itself.
b. It has the capability of providing real time structural health monitoring and damage locations can be identified.

However one of the interesting aspects of AE monitoring is that existing defects cannot be detected unless they are actively releasing energy. Therefore, if a crack or other defect is not growing (for example a crack that is self-arresting), AE activity will not be detected from that crack unless other sources are present, such as friction (also referred to as fretting). A schematic of AE mechanism is shown in FIG. 1.

To obtain a better understanding of the process occurring in a material, the basic parameters of an AE signal must be studied in combination to draw proper conclusions. ASTM E1316 is referenced for the definitions of AE parameters. Some of the AE parameters are defined below:

Hit—It is defined as the detection and measurement of an AE signal on and individual sensor channel.

Event—A local material change that gives rise to an acoustic emission. A single event can result in a number of hits.

Voltage Threshold—The voltage level on an electronic comparator such that signals with amplitudes larger than this level will be recognized. The threshold can be user defined or automatic floating, which is an important feature that helps to minimize noise.

Amplitude—It is defined as the magnitude of a peak voltage of the largest excursion attained by the signal waveform form a single emission event. Amplitude is measured in decibels (dB). Amplitude is obtained from the signal voltage by the below mentioned equation:

$$A = 20 log \frac{V}{Vref} \qquad ( Eq . 1 )$$

where,

V—Voltage of peak signal

V_{ref}—Reference voltage, dependent on the type of sensor

Duration—Defined as the time between AE signal stat and AE signal stop. In other words it is the time from the first threshold crossing to the last threshold crossing and is usually measured in micro seconds (μs).

Rise Time—It is defined as the time between AE signal start and peak amplitude of that signal and is measured in micro seconds (μs).

Signal Strength—It is defined as the measured area of the rectified AE signal with units measured in volt-sec. The formula for signal strength is given in Equation 2.

$$S = \frac{1}{2} [ {\int }_{t 1}^{t 2} {f}_{+} ( t ) \cdot d t + {\int }_{t 1}^{t 2} {f}_{-} ( t ) \cdot d t ] \qquad ( Eq . 2 )$$

Where, S is signal strength, f₊ and f₋ is positive and negative signal function.

Signal Energy—It is the energy contained in a acoustic emission burst signal, an is usually measured in joules.

Count—It is the number of times the acoustic emission signal exceeds a preset threshold during any selected portion of the test, and count rate is the number of counts during a fixed period of time.

Frequency—The number of cycles per second of an AE signal is known as its frequency, typically with units of kHz. Frequency is not a constant value for any hit. Commonly the average frequency is reported.

Root Mean Square (RMS)—Defined as the rectified time averaged AE signal, measured on a linear scale and reported in volts. RMS is the measure of continuously varying AE signal voltage.

The above recorded parameters of AE can be filtered for noise removal and analyzed using some of the damage quantification methods mentioned below:

a. Intensity Analysis

b. Felicity and Kaiser Effect

c. Calm ratio vs. load ratio

d. Peak Cumulative Signal Strength Ratio

e. Relaxation Ratio

f. b-value and lb-value

### SUMMARY

Objects and advantages of the invention will be set forth in part in the following description, or may be obvious from the description, or may be learned through practice of the invention.

Methods are provided for detecting damage in a post-tensioned concrete specimen due to corrosion. In one embodiment, the method comprises mounting at least two piezoelectric sensors onto the surface of the post-tensioned concrete specimen; receiving acoustic emission signals at the piezoelectric sensors; recording the acoustic emission signals with associated parameters and waveforms; and filtering the data for example using Root Mean Square data and the average frequency data.

The method can further include determining the level of damage in the post-tensioned concrete specimen due to corrosion via an Intensity Analysis chart and/or determining the location of damage in the post-tensioned concrete specimen due to corrosion via an Intensity Analysis.

Other features and aspects of the present invention are discussed in greater detail below.

### BRIEF DESCRIPTION OF THE DRAWINGS

A full and enabling disclosure of the present invention, including the best mode thereof to one skilled in the art, is set forth more particularly in the remainder of the specification, which includes reference to the accompanying figures.

FIG. 1 shows a schematic of AE mechanism.

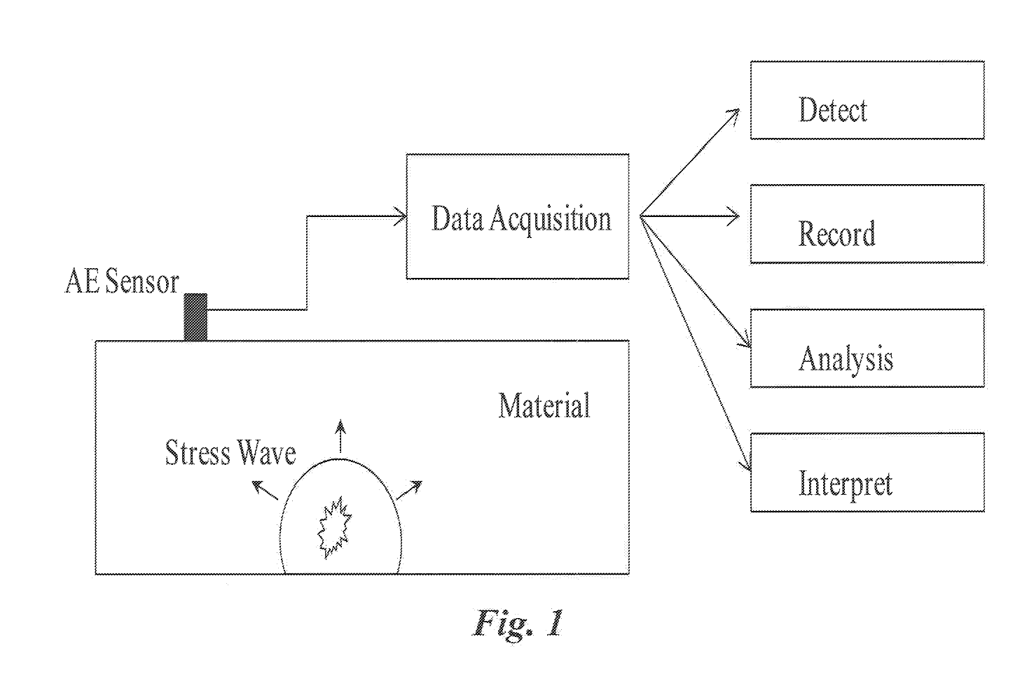

FIG. 2 shows a schematic of the experiential setup according to one embodiment of the present invention.

FIG. 3 shows plots of the half-cell potential [HCP] with respect to time and cumulative signal strength [CSS] with respect to time for each external PT specimen.

FIG. 4 shows plots of Duration vs. Amplitude of each external PT specimen.

FIG. 5 shows plots of Amplitude vs. Time of each external PT specimen.

FIG. 6 shows plots of HCP with respect to time and CSS with respect to time for each internal PT specimen.

FIG. 7 shows plots of Duration vs. Amplitude of each individual internal PT specimen.

FIG. 8 shows plots of Amplitude vs. Time of each individual internal PT specimen.

FIG. 9 shows plots of the Intensity Analysis for the external PT specimens.

FIG. 10 shows plots of the Intensity Analysis for the internal PT specimens.

FIG. 11 shows an intensity analysis chart for corrosion quantification.

### DETAILED DESCRIPTION

Reference now will be made to the embodiments of the invention, one or more examples of which are set forth below. Each example is provided by way of an explanation of the invention, not as a limitation of the invention. In fact, it will be apparent to those skilled in the art that various modifications and variations can be made in the invention without departing from the scope or spirit of the invention. For instance, features illustrated or described as one embodiment can be used on another embodiment to yield still a further embodiment. Thus, it is intended that the present invention cover such modifications and variations as come within the scope of the appended claims and their equivalents. It is to be understood by one of ordinary skill in the art that the present discussion is a description of exemplary embodiments only, and is not intended as limiting the broader aspects of the present invention, which broader aspects are embodied exemplary constructions.

As discussed above, corrosion of prestressing strands in post-tensioned structures is a slow process. However this process is accelerated due to ingress of chlorides or excess chlorides in the grout mix. Thus, the ingress of chlorides into Post-Tensioned [PT] concrete structures is a leading cause of corrosion of the prestressing strands, which reduces the strength, durability, and service life and may even result in catastrophic failure. However, visual inspection of post-tensioned bridges is difficult and they often lack a provision for electrochemical measurements to determine the corrosion levels in prestressing strands. As such, a structural health monitoring [SHM]method is needed to evaluate the damage in the prestressing strands. Acoustic Emission [AE] is one such method that may offer a solution. Research has shown that AE can be effectively used to detect corrosion in concrete structures. However, there is always a hindrance to AE signals due to thick concrete cover and the plastic ducts. Appropriate filters for AE need to be developed to minimize noise and retain corrosion data in realistic applications.

Previous research has largely focused on the use of AE to detect corrosion of passive reinforcement, and some studies have focused on prestressing applications. This paper investigates the use of AE to detect corrosion in active prestressing strands in a setting that is representative of both internal and external bonded post-tensioning.

Here, methods and systems using Acoustic Emission [AE] as a Structural Health Monitoring [SHM]method/system are generally provided to be employed to investigate corrosion in post-tensioned bridges constructed using both internal and external methods of post-tensioning. Through these methods, intensity analysis of AE data can be utilized to access/identify the damage in the PT specimens by categorizing according to the levels of corrosion present. Thus, AE can be successfully implemented to detect, monitor, and quantify corrosion levels in PT concrete structures.

Stress waves are caused by micro-crack formation associated with the expansive corrosion process and travel within the strand and grout medium. These stress waves are readily detected by piezoelectric sensors (referred to as Acoustic Emission sensors) in the appropriate frequency range (approximately between 10 and 100 kHz). For the case of external PT systems, the AE sensors can be simply affixed to the surface of the duct with ultrasonic or other couplant. For the case of internal PT systems, the AE sensor is simply mounted to the surface of the concrete using similar couplant. Acoustic emission signals (those received from the AE sensors) can be characterized by parameters such as amplitude, duration, rise time, energy, average frequency, Root Mean Square (RMS, which is defined as the rectified time averaged AE signal, measured on a linear scale and reported in volts. RMS is the measure of continuously varying AE signal voltage.) (ASTM E1316 2006), and signal strength (which is defined as the measured area of the rectified AE signal and reposted in volt-sec). These parameters are to some degree dependent on the magnitude and directionality of the source. The recorded AE parameters are plotted using specialized software (AEWin, Mistras Group, Inc.) to better understand the patterns and to aid in the development of filters for minimization of noise in the dataset due to reflections and other potential sources.

Two parameters were selected for data filtering; RMS and average frequency. After analysis of AE parameters during the weeks corresponding to when HCP values in the specimens (both internal and external PT) went below −250 mV, it was observed that average frequency and RMS showed similar tendencies, and hence these filters were chosen to minimize noise. In one embodiment, the filters for acceptance limits of the raw AE data are for RMS (V) between 0.0002 and 0.0004 and for Average Frequency (kHz) 30 to 100.

### Examples

To evaluate the potential of acoustic emission monitoring for this application, long term corrosion monitoring tests were performed on specimens that were representative of internal and external post-tensioning methods of construction. Corrosion was induced in the specimens by adding chlorides or by performing wet-dry cycling with NaCl solution. The corrosion process was monitored by half-cell potential measurements [HCP] and acoustic emission [AE]. Results show that AE has the ability to detect and quantify corrosion initiation, propagation, and cracking in the PT specimens with the same accuracy as HCP measurements. Further intensity analysis of AE data shows that the damage in the PT specimens can be categorized according to the levels of corrosion present. This investigation demonstrates that AE can be successfully implemented to detect, monitor, and quantify corrosion levels in PT concrete structures, and that AE is a promising SHM and assessment method to detect corrosion in the absence of regular electrochemical techniques.

Eight specimens (including two control specimens) were fabricated; four internal and four external PT. Wet-dry cycling was performed on the external PT specimens and additional chlorides were added to the internal PT specimens. Corrosion in each specimen was monitored with two AE sensors and an embedded sliver chloride reference electrode. The AE data was validated by electrochemical measurements such as half-cell potentials [HCP]. HCP measurements were taken on a weekly basis and AE activity was continuously recorded for four months. Raw AE data was filtered and correlated to HCP measurements to investigate the ability of AE to monitor and assess the state of corrosion damage.

### EXPERIMENTAL SETUP

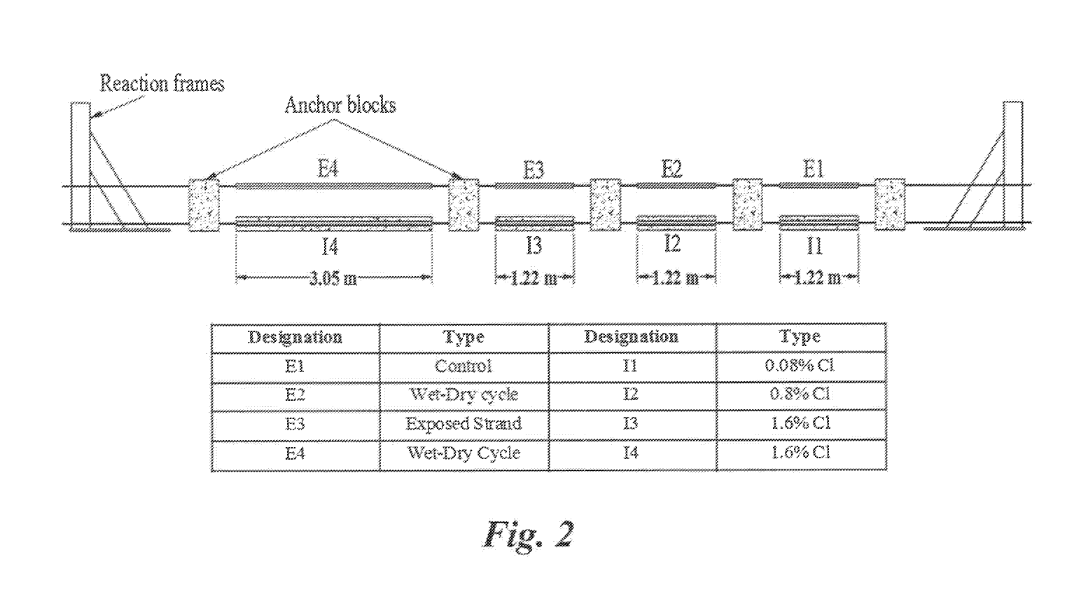

The experimental setup was designed to be representative of both external and internal PT systems. Two 12.5 mm (½ in.) diameter seven wire strands of length 19.5 m (58 ft.) were stressed and supported on two steel reaction frames. A total of eight specimens were created around the strands, of which four specimens represent external PT and the remaining four represent internal PT. Anchor blocks made of concrete were cast around the strands and in-between specimens for safety in the event of strand failure due to corrosion. FIG. 2 is a schematic of the experiential setup.

Externally Post-Tensioned Specimens:

Four external PT specimens were created by encasing a seven wire 12.5 mm (½ in.) diameter low relaxation strand in a HDPE duct of smooth external surface with a diameter of 60.3 mm (2⅜ in) and thickness of 6 mm (¼ in.). The strand was stressed to 0.7 fₚᵤ (guaranteed ultimate strength, fₚᵤ=270 ksi) through the application of an axial tensile force. A cement based grout manufactured by BASF (BASF Masterflow 1205) was used for grouting. This grout is a special grout specifically for PT structures and is also used by many DOT's across the U.S. A water quantity of 1,100 ml was added for every 4.54 kg (10 lbs.) of grout. This mix provides good consistency and fluidity for pumping into ducts. For these specimens no additional chlorides were added to the grout mix. Of the four specimens one measured 3.05 m (10 ft.) long and the remaining three were 1.22 m (4 ft.) long. One of the smaller length specimens served as control specimen, E1.

Specimen E3 measured 1.22 m (4 ft.) in length and had the strand exposed to the environment mimicking an air void and corrosion under environmental conditions. To achieve this, a small section of size 300 mm×25 mm (12 in.×1 in.) was cut from the HDPE duct and a small piece of wood was placed, so that when the duct is grouted the grout will fill the duct leaving the strand exposed. The exposed strand specimen, E3, was left exposed to the atmosphere for the first 45 days, after which a small quantity of 2% NaCl solution was sprayed only once on the strand to induce corrosion. The two remaining specimens, E2 with a length of 1.22 m (4 ft.) and E4 with a length of 3.05 m (10 ft.), had 300 mm (1 ft.) long, 25 mm (1 in.) wide and 12.5 mm (½ in.) deep grooves to facilitate wet-dry cycling. These wet-dry specimens were subjected to 3 days wet and 4 days dry cycles. Initial wet-dry cycles were started with a 2% NaCl solution. This NaCl solution was maintained for four weeks and then the percentage of NaCl was increased by 2% every week until it reached 10% and was maintained at that level thereafter. A silver chloride (AgCl) reference electrode was embedded into each duct for half-cell potential measurements.

Internally Post-Tensioned Specimens:

For the internal PT specimens, the lengths were similar to the external PT specimens. The strand was encased using corrugated poly propylene [PP] grouted ducts with 60.3 mm (2⅜ in.) diameter and 2 mm ( 1/10 in.) thickness, as opposed to smooth HDPE duct which was used for the external PT specimens. The advantage of having a corrugated duct is that it provides a better bond between the concrete and the duct. The type of grout and water content for the mix remained the same as for external PT specimens.

Chlorides were added in varying percentages in all four specimens. The shorter specimens I1, I2 and I3 had 0.08%, 0.8% and 1.6% chlorides by weight of grout respectively, whereas the longer specimen I4 had 1.6% chlorides by weight of grout. Roughly 16 kg (35 lbs.) of dry grout was required to fill each of the shorter specimens and about 36 kg (80 lbs.) of dry grout was required to fill the longer 3.05 m (10 ft.) specimen. An air pressure powered pump (ChemGrout CG050) was used to pump the grout into the duct which had an output pressure of 1.55 MPa (225 psi). Concrete was then placed around the ducts with a cross-section of 165 mm×165 mm (6½% in.×6½% in.). The cross-section was based on the knowledge that reinforcement is generally located within a structure with a minimum cover of 50 mm (2 in.).

Table 2.1 shows the mix design used for the concrete. Silver chloride (AgCl) reference electrodes were embedded in the grouted ducts for the purpose of half-cell potential [HCP] measurements.

TABLE 2.1
Concrete mix design for internal PT specimens
Concrete MixDesign

| | |
|---|---|
| Cement | 370 kg/m³ |
| Fine Aggregates | 675 kg/m³ |
| Coarse Aggregates | 1240 kg/m³ |
| Water | 140 kg/m³ |

Acoustic Emission Equipment:

Basic terminology and concepts of AE are discussed above. Stress waves associated with corrosion travel within the strand and grout medium and can be detected by a piezoelectric sensor attached to the surface of the duct or the concrete cover. AE signals can be classified by parameters such as amplitude, duration, rise time, energy, average frequency, RMS, and signal strength that are unique and depend on the magnitude and directionality of the source. The recorded AE parameters can be plotted using special software such as AEWin (Mistras Group, Inc.) to understand the pattern and to aid in the development of filters to minimize noise and retain meaningful data.

Each of the specimens was monitored with two acoustic emission (AE) resonant sensors, 55 kHz with an integral 40 dB pre-amplifier (R6i), (Mistras Group, Inc.). The sensors were strategically located on the specimens to pick up corrosion activity. The sensors were connected to a 16-channel data acquisition system (Sensor Highway II, Mistras Group, Inc.). For the internal PT specimens, one AE sensor was positioned at the center of the top surface of the specimen while the other sensor was positioned on one face of the cross-section between the strand and the grout.

For the external PT specimens, the shorter specimens had a sensor located at 300 mm (1 ft.) from each corner while the longer external PT specimen had sensors located at 910 mm (3 ft.) from corner. The test threshold was set to 40 dB for all channels.

### Results and Discussions

### Externally Post-Tensioned Specimens

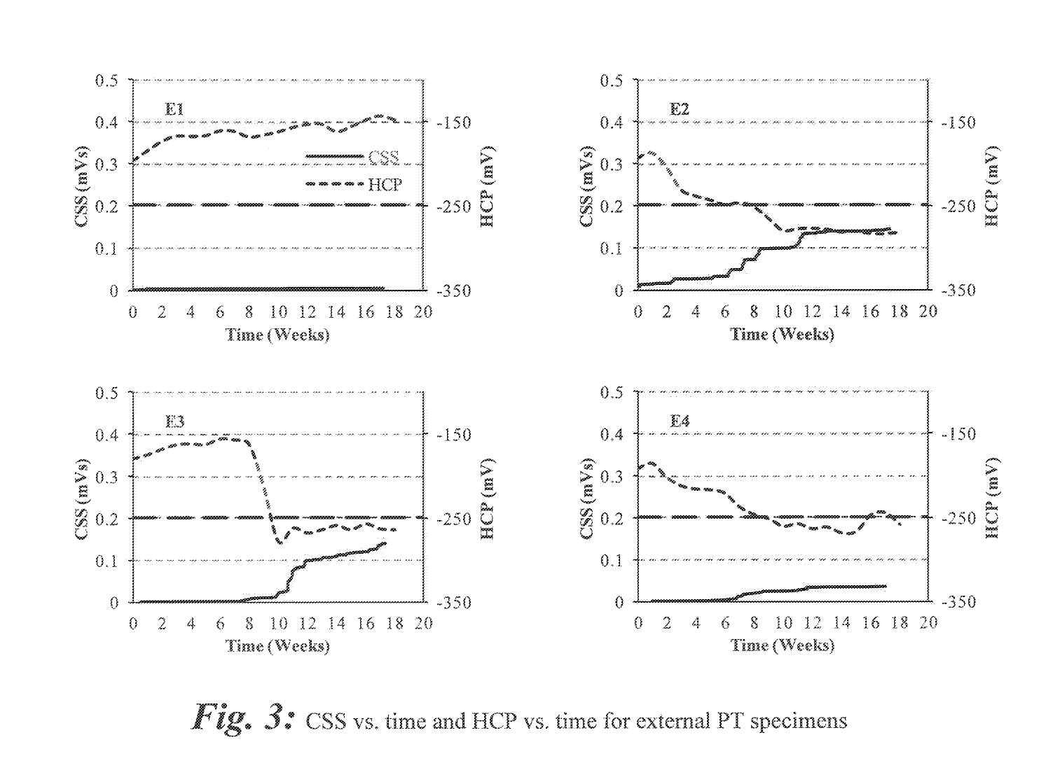

The ASTM criterion for corrosion of steel in concrete for a silver chloride (AgCl) reference electrode is given in Table 2.2. The half-cell potential [HCP] with respect to time and cumulative signal strength [CSS] with respect to time for each external PT specimen are plotted in FIG. 3. The HCP measurements were taken with an impedance voltmeter on a weekly basis. In specimen E1, the control specimen, the HCP measurements indicate that corrosion did not initiate. This agrees with the AE data as minimal AE activity was collected over the span of the test.

TABLE 2.2
ASTM corrosion criteria for AgCl reference electrode (Broomfield, 2007)

| Potential Against AgCl Electrode | Corrosion Condition |
|---|---|
| >−100 mV | Low Risk (10% probability of corrosion) |
| −100 to −250 mV | Intermediate corrosion risk |
| <−250 mV | High Corrosion risk (90% probability) |
| <−400 mV | Severe Corrosion Damage |

In specimens E2 and E4, the wet-dry cycles were initiated five days after installing the AE sensors on the specimens. With an initial NaCl solution concentration of 2% it was observed that corrosion initiated about one week from the beginning of wet-dry cycling, and the half-cell potentials of the specimens went below the threshold of −250 mV by the eighth week of wet-dry cycling. As the NaCl concentration was raised, the corrosion level increased rapidly, which is seen in the half-cell potential measurements as these values went on to become more negative and the recorded AE had significant jumps in signal strength when the NaCl concentration was raised. The AE decreased in the later stages, which may be associated with lower rates of corrosion due to a thin protective layer on the surface of the wires precluding further corrosion of the inner layers. For more corrosion to occur within a reasonable period of time, the salt concentration must be raised.

E3 is the specimen with the strand exposed to air. Since there are no chlorides in the grout or provision for wet-dry cycling, there was no corrosion activity in the beginning. After 45 days, a small amount of 2% NaCl solution was sprayed on the exposed strand to initiate corrosion. Within two weeks there were visible signs of corrosion, and by the fourth week the HCP measurements fell below the threshold of −250 mV. During this time, the HCP values plummet and there is a related sudden rise in AE signal strength. The corrosion of the specimens continued even in the absence of the NaCl solution. This can be explained by the work of Feng et al. [25] where it is stated that due to stresses developed in the steel, the rate of re-passivation is much slower and therefore the steel is more susceptible to corrosion. Regular visual inspection of this specimen confirmed this, and it was also apparent in the HCP and AE measurements. Visible signs of corrosion were observed in this specimen. A closer look also revealed grout cracking around the strand.

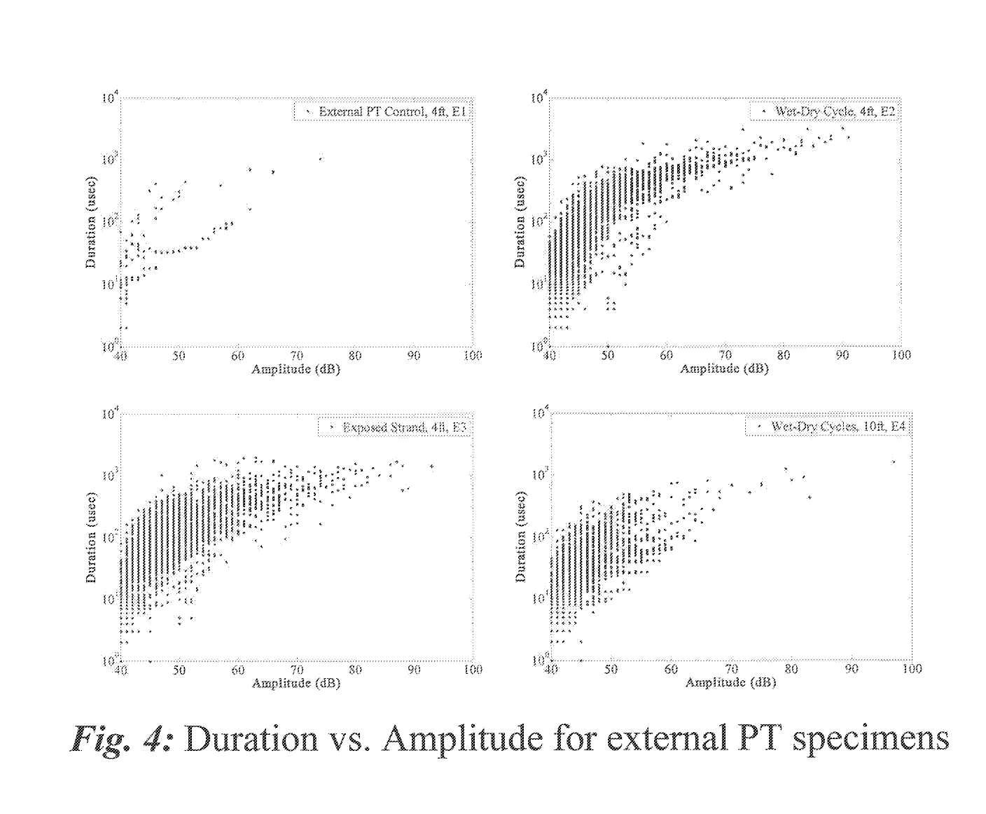

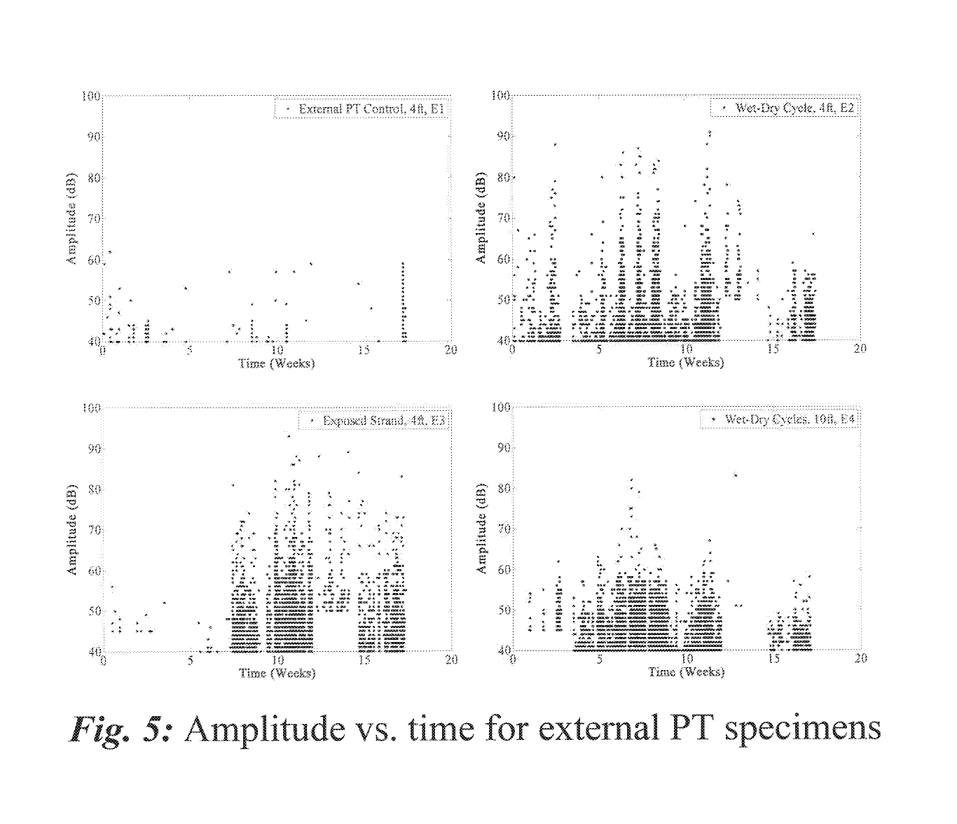

FIGS. 4 and 5 are plots of Duration vs. Amplitude and Amplitude vs. Time of each external PT specimen, respectively. From these plots, it can be seen that for specimen E1 there are very few hits of low amplitude and low duration, indicating no active corrosion which is also validated by HCP measurements. The plots for specimens E2, E3, and E4 show there is high AE activity that can be related to active corrosion and is validated by HCP measurements. One interesting feature to notice is, specimens E2 and E3 show a significant number of hits above 60 dB while that is not the case for E4 wherein there are just a few hits higher than 60 dB. Visual inspection of E2 and E3 show grout cracking but no cracking is seen in E4. Therefore it is hypothesized that the grout cracking due to corrosion in this case may be associated with hits of amplitude greater than 60 dB.

### Internally Post-Tensioned Specimens

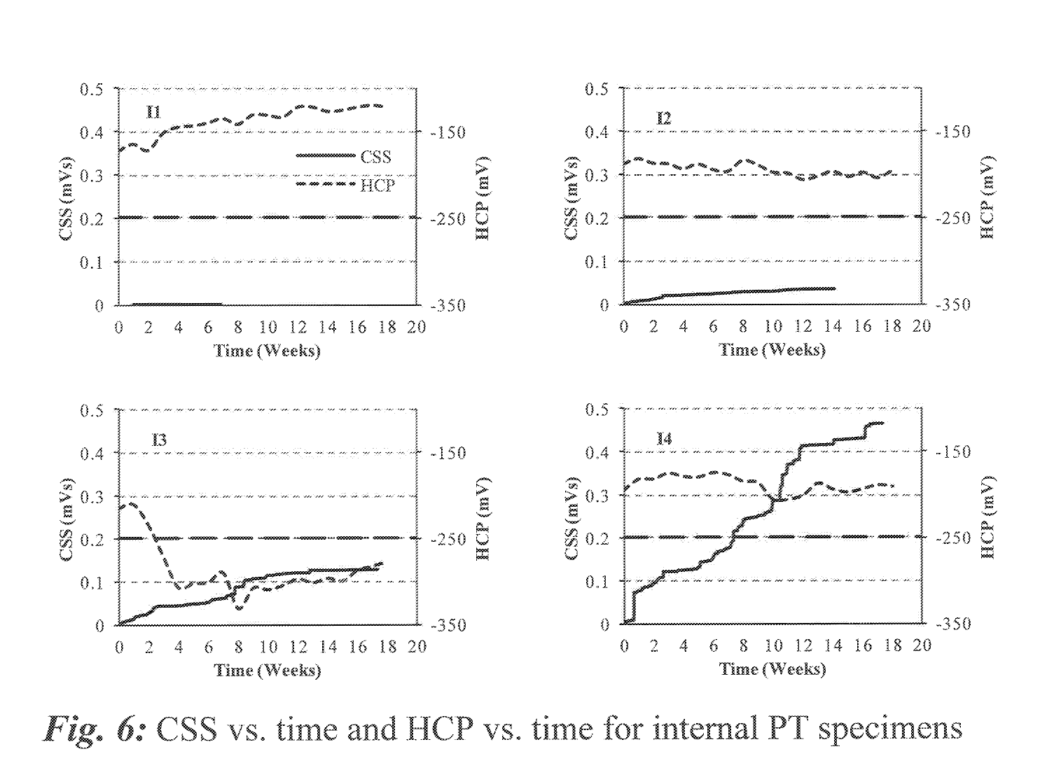

HCP with respect to time and CSS with respect to time for each internal PT specimen are shown in FIG. 6. For specimen I1 the HCP values are close to −120 mV, indicating a lack of active corrosion. The lack of corrosion activity is reflected in the lack of AE activity, which has less than 50 hits throughout the time the AE signals were recorded. With 0.8% chlorides, the HCP values for specimen I2 are close to −200 mV which is slightly above the threshold of −250 mV, implying that the corrosion level in this specimen is uncertain.

Specimens I3 and I4 vary only in their length, having the same chloride content. The HCP of specimen I3 shows corrosion initiated in the second week and this specimen reached the threshold HCP of −250 mV by the fourth week In the case of specimen I4, the HCP measurements are in the uncertain region running close to −200 mV. Analysis of the acoustic emission data such as CSS vs. Time, Amplitude vs. Time and Intensity Analysis indicates active corrosion in both specimen I3 and I4. Some potential reasons to explain the HCP measurements of specimen I4 are: uneven distribution of chlorides in the specimen, leading to lesser corrosion occurring in the region where the reference electrode is located; a lack of contact between the reference electrode and the strand or grout; internal air pockets; or a faulty reference electrode. One method to cross check the performance of this reference electrode in I4 is by measuring the potential between it and another reference electrode preferably silver chloride, but to perform this action the reference electrode has to be removed from the specimen first. This illustrates the advantage of using AE as an SHM method to detect corrosion in PT structures because conventional electrochemical measurements can sometimes be misleading. The advantage of HCP measurements is the simplicity of the technique; however it has been reported that HCP can show no corrosion when corrosion actually is occurring and vice-versa [26, 27].

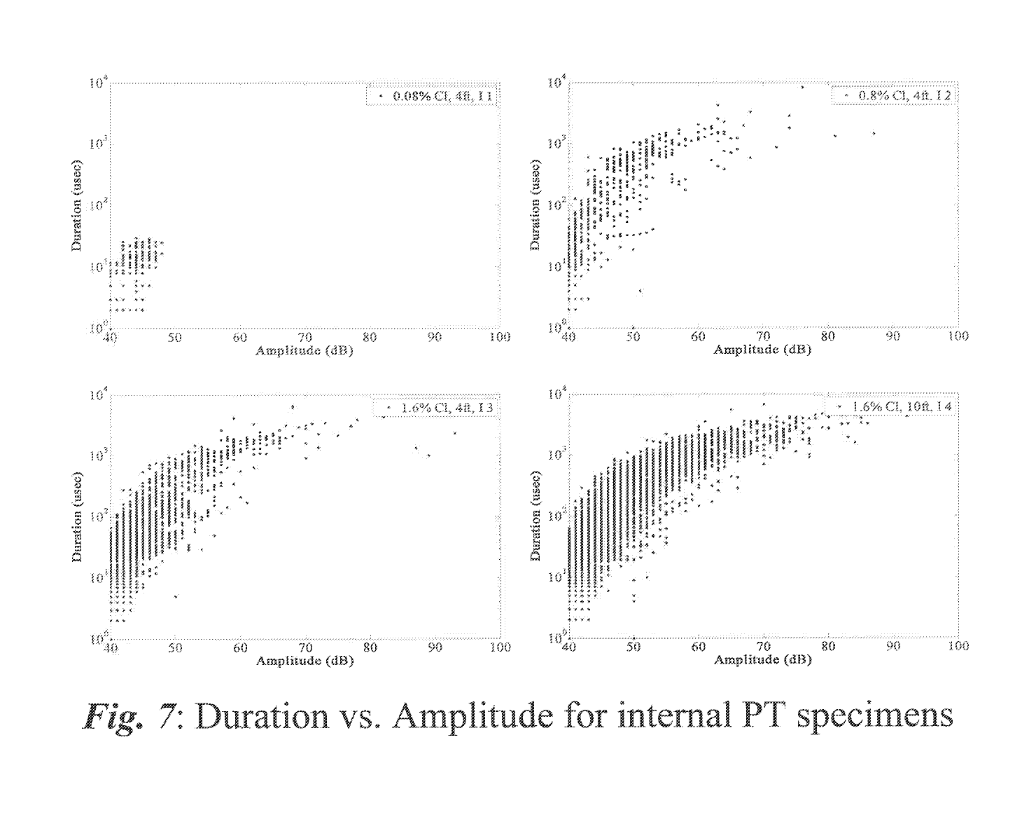

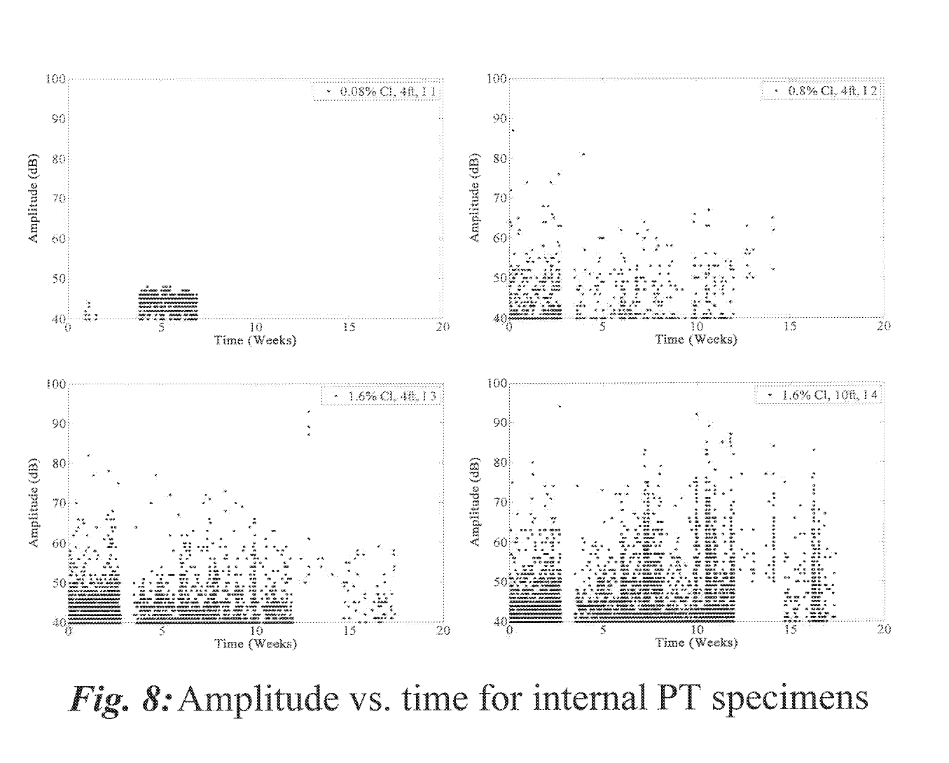

FIGS. 7 and 8 are plots of Duration vs. Amplitude and Amplitude vs. Time of each individual internal PT specimen respectively. Here it can be seen that the control specimen I1 has the least recorded AE activity indicating no active corrosion which is in accordance with HCP measurements. Specimen I3 and I4 have very high AE activity which can be related to corrosion, while specimen I2 has lesser AE activity as compared to I3 and I4 indicating lesser active corrosion, all of which is in accordance with HCP measurements.

### Intensity Analysis

Intensity Analysis is a damage quantification methodology for AE data. It is used to aid in the understanding of the degree of damage and is a statistically based method related to AE signal strength, which is used to compute Historic Index and Severity. Intensity Analysis is a plot of Historic Index on the x-axis and Severity on y-axis. Historic Index, H(t), is the measure of change of signal strength whereas Severity (Sᵣ) is an average of the largest 50 signal strength values. Equations to calculate Historic Index and Severity are:

$$\begin{matrix}H ( t ) = \frac{N}{N - K} \cdot ( \frac{{\sum }_{i = K + 1}^{N} {S}_{oi}}{{\sum }_{i = 1}^{N} {S}_{oi}} ) & ( Eq . 2.1 ) \\ {S}_{r} = \frac{1}{J} \cdot \{ {\sum }_{m = 1}^{J} {S}_{om} \} & ( Eq . 2.2 )\end{matrix}$$

where, N is the number of hits up to a time (t), Sₒᵢ and Sₒₘ are signal strength, and K and J are empirical constants based on material under consideration. The constants K & J depend on N, and for concrete are often given as a) K=N/A if N≦50; b) K=N−30 if 51≦N≦200; c) K=0.85 N if 201≦N≦500; d) K=N−75 if N≧501; e) J=0 for N<50; and f) J=50 for N≧50. To portray the level of damage, an Intensity Analysis chart is subdivided into regions where each region corresponds to a particular level of damage.

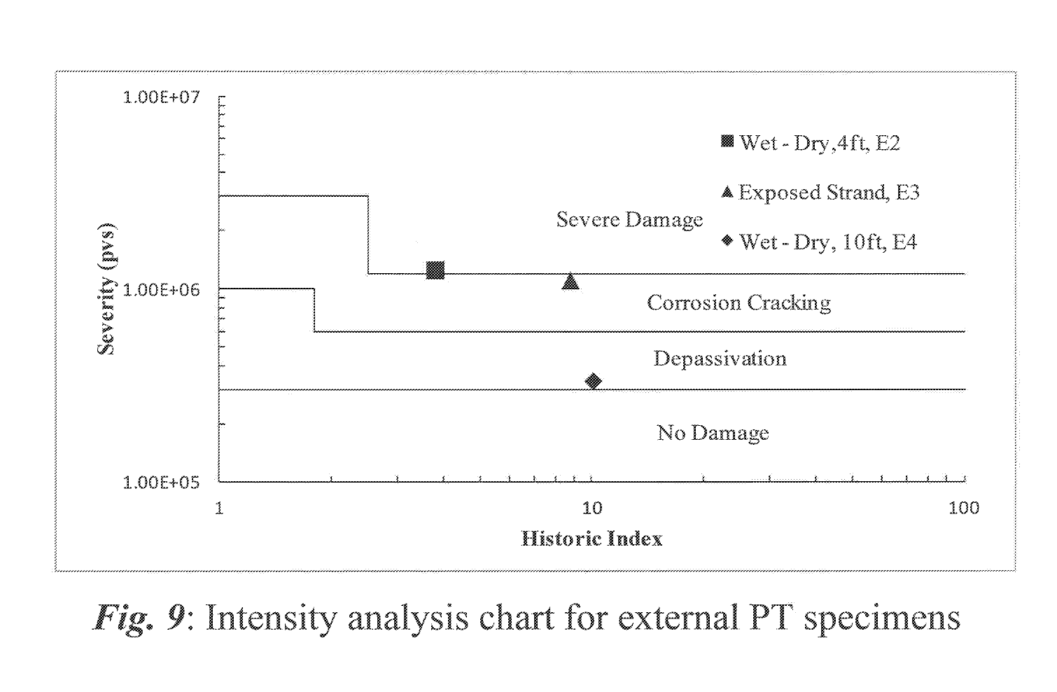

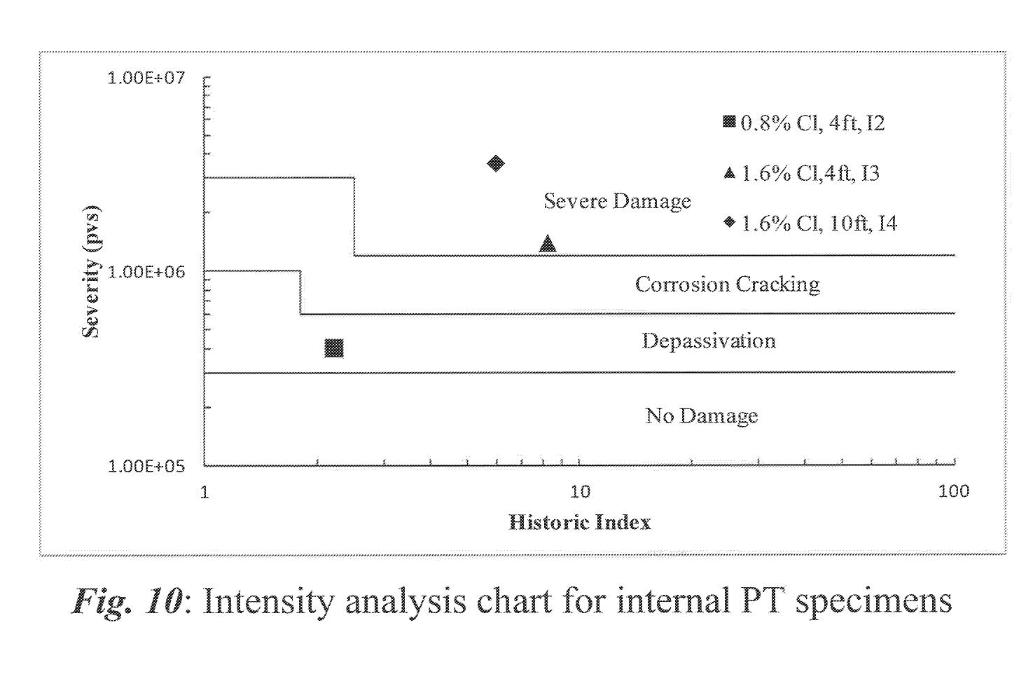

FIGS. 9 and 10 are the Intensity Analysis plots for the external and internal PT specimens respectively. Specimens E1 and I1 have no corrosion and the number of AE hits is less than 50, therefore the coordinates on the Intensity Analysis chart are at unity implying there is no damage. This corresponds with the HCP values. Specimens E2 and E3 recorded close to 4,500 hits each and are at the extremes of cracking and entering into the severe damage region, as verified by visual inspection of these specimens. Specimen E4 differs from specimen E2 only in length and recorded almost 2,000 more hits than E2, but still the signal strengths are much lower than E2, and on the intensity chart it falls in the de-passivation region. Although there was active corrosion in this specimen, there was not much grout cracking occurring around the corroded strand.

Specimen I2 plots in the de-passivation region, and specimens I3 and I4 plot in the severe damage region, in accordance with the HCP measurements except for I4. The possible reasons for improper HCP measurements for I4 have been previously discussed.

### CONCLUSIONS

Corrosion was induced in both internal and external PT specimens. Long term monitoring of corrosion was performed on the specimens by half-cell potential and acoustic emission monitoring. Conclusions are:
a) Acoustic Emission is not only successful in detecting and quantifying damage in PT structures due to corrosion, but may detect the corrosion process prior to the conventional electrochemical measurements. Importantly, it can be effectively used where there is no provision for electrochemical measurements.
b) Intensity Analysis proved to be useful to determine the degree of damage in the specimens. The results were in accordance with the HCP measurements.
c) Appropriate filters need to be utilized to minimize noise.
d) AE can be a very efficient monitoring method to detect corrosion in PT concrete structures in real time.

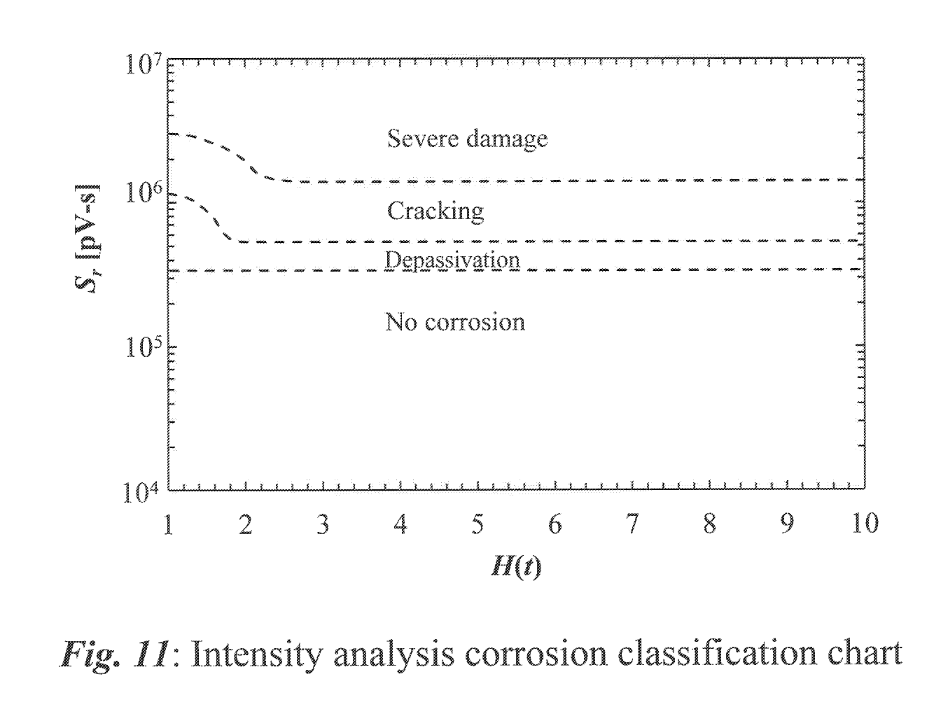

The Intensity Analysis chart for corrosion quantification is shown in FIG. 11 based on signal strength in units of pV-s. The boundaries of this chart were plotted based on an extensive study on small scale cracked specimens. The chart divides the corrosion damage into four categories based as shown in Table 3. By plotting the data point from a sample in FIG. 11, the corrosion damage in the specimen can be quantified.

TABLE 3
Corrosion Damage Classification

| Assessed | |
|---|---|
| condition | Comments |
| No damage | At this level the steel is still in the passive condition and no |
| | corrosion damage occurred |
| Depassivation | At this level corrosion has just initiated with sectional mass |
| | loss less than 15% |
| Cracking | Refers to the level at which cracks due to corrosion started |
| | to form and the sectional mass loss is less than 21% |
| Severe damage | More cracks form and the sectional mass loss exceeds 21% |

These and other modifications and variations to the present invention may be practiced by those of ordinary skill in the art, without departing from the spirit and scope of the present invention, which is more particularly set forth in the appended claims. In addition, it should be understood the aspects of the various embodiments may be interchanged both in whole or in part. Furthermore, those of ordinary skill in the art will appreciate that the foregoing description is by way of example only, and is not intended to limit the invention so further described in the appended claims.

What is claimed:

1. A method of detecting damage in a post-tensioned concrete specimen due to corrosion, the method comprising:
mounting at least two piezoelectric sensors onto the surface of the post-tensioned concrete specimen;
receiving acoustic emission signals at the piezoelectric sensors;
recording the acoustic emission signals with associated parameters and waveforms; and
filtering the data for example using Root Mean Square data and the average frequency data.

2. The method of claim 1, further comprising:
determining the level of damage in the post-tensioned concrete specimen due to corrosion via an Intensity Analysis chart.

3. The method of claim 1, further comprising:
determining the location of damage in the post-tensioned concrete specimen due to corrosion via an Intensity Analysis.
